(19) United States
(12) Patent Application Publication
(10) Pub. No.: US 2014/0243822 A1
(43) Pub. Date: Aug. 28, 2014

(54) RF Surgical Resection Instrument Having a Resection Loop for Removal of Pathological Tissue

(71) Applicants: Günter Farn, Tubingen (DE); endox Feinwerktechnik GmbH, Bad Urach (DE)

(72) Inventors: Günter FARIN, Tubingen (DE); Matthias HERNIK, Bad Urach (DE)

(73) Assignees: Gunter Farin, Tubingen (DE); endox Feinwerktechnik GmbH, Bad Urach (DE)

(21) Appl. No.: 14/268,028

(22) Filed: May 2, 2014

Related U.S. Application Data
(63) Continuation of application No. PCT/EP2012/071629, filed on Oct. 31, 2012.

(30) Foreign Application Priority Data
Nov. 3, 2011 (DE) 10 2011 085 721.4

(51) Int. Cl. A61B 18/14; A61B 17/221

(52) U.S. Cl. CPC A61B 18/1445; A61B 17/221; A61B 2018/00607
USPC 606/46

(57) ABSTRACT

An asymmetrical opening RF surgical resection loop comprises a short electrically non-insulated loop section and a long electrically insulated loop section, where the distal ends of the loop sections are interconnected. Furthermore, a manipulation wire guided in an insulated catheter and displaceable in the longitudinal direction thereof is connected to this loop section in order to slide the long loop section out from the catheter. The short loop section is withdrawn from the catheter by the long loop section. An insulated stop wire is connected to the short loop section and is also pulled out a predefined length from the catheter by this, where at least the pulled out section of the stop wire is electrically insulated. By means of an entrainer device, the stop wire and the short loop section connected to this is withdrawn into the catheter upon withdrawal of the manipulation wire.

### PRIORITY CLAIM

This application is a continuation of pending International Application No. PCT/EP2012/071629 filed on 31 Oct. 2012, which designates the United States and claims priority from German Application No. 10 2011 085 721.4 filed on Nov. 3, 2011. The contents of both of these applications are incorporated by reference in their entireties.

### BACKGROUND OF THE INVENTION

1. Field of the Invention

The invention relates to RF surgical resection instruments having asymmetrical resection loops such as are used, for example, for the endoscopically controlled resection of polyps or flat lesions of the mucosa of the gastro-intestinal tract.

2. Description of Relevant Art

An important requirement for endoscopically controlled resection, in particular of large (>2 cm) polyps or large (>2 cm) flat lesions of the mucosa of the gastro-intestinal tract, is the adherence to relevant requirements here on the part of oncology, namely, resection in sano, i.e. as far as into healthy tissue, and pathology, namely resection as far as possible en-bloc, i.e. in one piece, and below the first third of the submucosa (sm1) or as close as possible to the muscularis propria.

With the RF surgical resection loops corresponding to the prior art, so-called polypectomy snares, these requirements of oncology and pathology can only be satisfied with difficulty or not satisfied at all in cases of large polyps (>2 cm) or lesions (>2 cm) of the mucosa and, in particular, not when, prior to the RF surgical resection, electrically conductive liquids such as, for example, physiological NaCl solution is injected into the submucosa under these polyps or lesions in order to distance these polyps or lesions from the muscularis propria located thereunder, which must not be thermally damaged during the RF surgical resection. This problem results from the physics of endoscopic polypectomy (EPE). Conventional polypectomy loops require an RF current of about 0.5 Aeff for a low-delay incision. RF generators of available RF surgical instruments can generate a maximum of 2 Aeff. Polyps or lesions having a circumference of about 4 cm or a diameter of about 1.3 cm can be resected herewith en-bloc with negligible incision delay. In cases of larger polyps or lesions, longer incision delays must be risked with the risk of thermal damage to the muscularis propria and perforation of the organ wall thereby caused. For this reason, in practice, polyps or lesions inter alia are only resected en-bloc up to a maximum diameter of 2 cm, where a still-acceptable incision delay is risked.

Polypos or lesions larger than 2 cm in diameter are either removed with polypectomy loops in several pieces (piece meal) or by the method of endoscopic submucosa dissection (ESD) en-bloc. Since piecemeal resection does not meet the requirements of pathology and only satisfies the requirements of oncology to a limited extent (high recurrence rate), and because ESD is technically very demanding and also very time-consuming and consequently expensive, alternative resection methods and suitable resection instruments for this are being sought.

WO 2011/012616 A2 discloses, inter alia, an asymmetric monopolar snare with which tissue to be removed can be ensnared and separated by RF surgery, comprising a short electrically non-insulated snare section which can be applied as RF surgical active electrode and a long electrically insulated snare section which cannot be applied as RF surgical active electrode, where the short and the long snare section are interconnected at their distal ends and where the long snare section is connected to a manipulation wire at its proximal end, with which this snare section can be drawn into a catheter and pushed out from this again, and the short snare section is connected to a stop wire at its proximal end so that the short snare section can only be pulled out from the distal end of the catheter as far as a stop, whilst the long snare section can be pushed further out from the distal end of the catheter, whereby the two snare sections form an opened snare.

This asymmetric monopolar snare is beset with two mutually contradictory problems. On the one hand, the short snare section should be as short as possible so that the RF current required for the RF surgical cutting, in particular during the incision phase and/or when an electrically conductive liquid, for example physiological NaCl solution is injected submucosally under the mucosa to be resected, is as far as possible smaller than the maximum RF current which can be generated by available RF generators, and on the other hand, the short snare section should be as long as possible so that the snare opens with the smallest possible shear when pushing out the long snare section.

In addition, in special cases of application it can be problematical to press this snare sufficiently firmly and flat and in a controlled fashion against the organ wall during the ensnarement and/or during the resection.

In addition, if this snare is not already ensnared around tissue, it can cut in an unintended direction.

### SUMMARY OF THE INVENTION

The embodiments are based on the object of providing an RF surgical resection instrument comprising a loop as well as a resection loop, also called snare, which is not beset with the problems listed above.

In an embodiment, an RF surgical resection instrument comprises
an asymmetrically opening and closing resection loop further comprising a short electrically non-insulated loop section (RF surgical cutting and/or coagulation electrode or active electrode) having a proximal and a distal end and a long electrically insulated loop section having a proximal and a distal end, wherein the two loop sections consist of the same or of different material and their distal ends are mechanically and/or distally connected to one another,
a rigid or flexible catheter made of electrically non-conductive material having a proximal and a distal end,
a manipulation wire having a proximal and a distal end, which can be advanced inside the catheter in the axial direction from proximal to distal and can be withdrawn from distal to proximal and its distal end is connected mechanically and/or electrically to the proximal end of the long loop section or which is formed as an extension of the proximal end of the long loop section, whereby the short loop section is withdrawn from the distal end of the catheter when advancing the long loop section,
a stop device further comprising a stop wire having a proximal and a distal end, wherein the distal end of the stop wire is connected to the proximal end of the short loop section, a stop element which is movable relative to the catheter on the proximal end of the stop wire and
   a stop element which is fixed relative to the catheter inside the catheter, on which the stop element impacts when withdrawing the short loop section,
an electrical insulation at least on a distal section of the stop wire or on the entire stop wire, wherein a distal electrically insulated section of the stop wire projects from the distal end of the catheter when the stop wire impacts on the stop device.
an entrainer element further comprising an entrainer element on the manipulation wire and an entrainer element on the stop wire.

When the loop is completely opened, the proximal end of the long loop section must project sufficiently far into the distal end of the catheter or at least the distal end of the manipulation wire must be electrically insulated because otherwise hot electrical arcs here can also damage the distal end of the catheter.

The embodiments are based on comprehensive technical and scientific investigations of a plurality of RF surgical loops and resections carried out therewith. Some of the results of these investigations have already been implemented in the RF surgical resection loops disclosed in WO 2011/012616 A2. In order to be able to also resect large polyps and large flat lesions of the mucosa endoscopically using resection loops en-bloc and close to the muscularis propria, an asymmetric resection loop is proposed in which the long loop section is electrically insulated and the short loop section is not electrically insulated. When the asymmetric loops are opened, the electrically non-insulated loop section according to WO 2011/012616 A2 extends from the distal end of the catheter to the distal end of the loop or the point where it is connected to the electrically insulated long loop section. This electrically non-insulated loop section serves as RF surgically active electrode and is generally also called cutting wire. The shorter the cutting wire, the smaller is the RF current required for the RF surgical cutting and in particular the RF current required for the shortest possible incision delay.

However, the electrically non-insulated loop section in the resection snares disclosed in WO 2011/012616 A2 cannot be made as short as would be expedient in particular in resections of polyps and flat lesions below which electrically conductive liquid, for example, physiological NaCl solution has been injected submucosally prior to the resection. Since, on account of the aforesaid requirements from oncology and pathology, this underinjection is becoming more and more standard even for smaller polyps and smaller flat lesions, it is also becoming necessary to make the non-electrically insulated loop section or cutting wire as short as possible. However, this conflicts with the fact that the shear force required to open asymmetrical resection loops is all the greater, the shorter is this cutting wire. From a critical length or shortness of the cutting wire, the loop can only be opened with very high shear force or no longer opened at all.

This problem is solved whereby when the asymmetrical resection loop is opened, a piece of the stop wire that is electrically insulated projects from the distal end of the catheter and thus together with the RF surgically optimally short cutting wire is part of the optimally short loop section with regard to shear force.

Since now during the RF surgical resection, not the cutting wire but the electrically insulated stop wire, touches the distal end of the catheter, the distal end of the catheter can neither by damaged by a hot cutting wire nor by electrical arcs which are unavoidable during RF surgical cutting.

### BRIEF DESCRIPTION OF THE DRAWINGS

In the following, the invention will be described by way of example, without limitation of the general inventive concept, on examples of embodiment and with reference to the drawings.

FIG. 1 shows an exemplary embodiment of a resection instrument.

FIG. 2 shows in detail an exemplary embodiment of the connection of the two loop sections at the proximal end of the resection loop.

FIG. 3 shows a spur at the distal end of the resection loop.

FIG. 4 shows a mandrel at the distal end of the resection loop.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

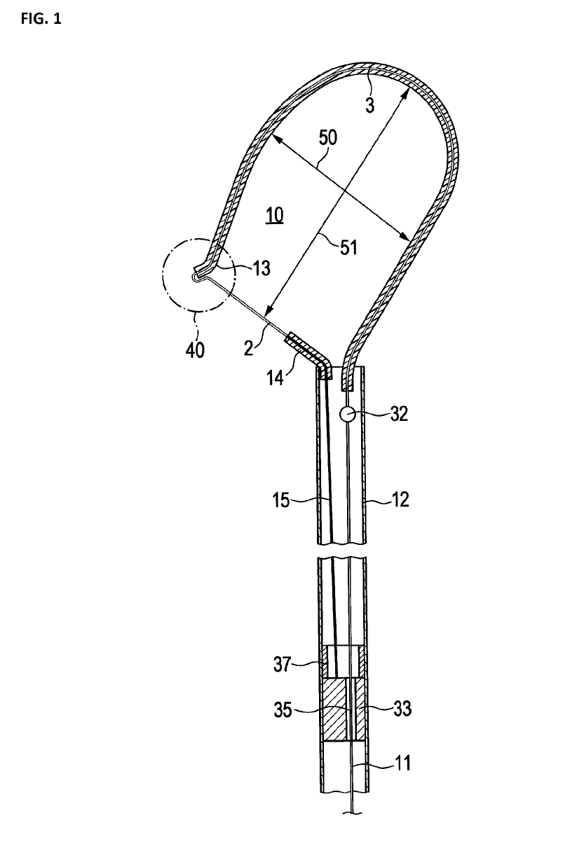

FIG. 1 shows an exemplary embodiment of an RF surgical resection instrument comprising an asymmetric resection loop 10, a catheter 12 and a stop device 33, 37, 15 as well as an entrainer device 32, 33, 15 and a manipulation wire 11 having a proximal and a distal end.

The asymmetrically opening and closing RF surgical resection loop 10 comprises a short, electrically non-insulated loop section 2 having a proximal end and a distal end as well as an electrically insulated long loop section 3 having a proximal and a distal end, which is surrounded by an electrical insulation 13. The long loop section 3 and the short loop section consist of the same or of different metal wires, preferably the long loop section 3 however consists of a spring-elastic metal wire and the short loop section 2 consists of flexible metal wire or flexible metal braid. The short loop section 2 and the long loop section 3 are mechanically and/or electrically interconnected at their distal ends.

The rigid or flexible catheter having a proximal and a distal end consists of electrically non-conductive material.

The manipulation wire consists of a metal wire which is preferably spring-elastic and torsionally rigid but can consist of the same material as the long loop section 3. The distal end of the manipulation wire 11 is connected to the proximal end of the long loop section 3.

The stop device comprises a stop 37 fixed in the catheter, a stop element 33 which is movable in the catheter and a stop wire 15 having a proximal and a distal end. The proximal end of the stop wire 15 is connected to the stop element 33 and the distal end of the stop wire is connected to the proximal end of the short loop section 2. The stop wire 15 and the short loop section 2 consist of the same or of different material.

The stop wire 15 is surrounded by an electrical insulation 14 at least at its proximal end section or over its entire length.

The stop device delimits the maximum length which the short, electrically non-insulated loop section 2 including a proximal electrically insulated end section 14 of the stop wire 15 during sliding of the long loop section out from the distal end of the catheter 12 in order to open the loop 10 by further sliding out the long loop section 3.

The shear force required to open an asymmetrical resection loop is greatest when the long loop section has pulled out the short loop section as far as its stop from the distal end of the catheter so that the section of the long loop section slid out from the distal end of the catheter is the same length as the short loop section. This phenomenon is not relevant in conventional asymmetric resection loops in which the long loop section is not electrically insulated and which have already been in use for more than 30 years, because in these resection loops, the long loop section is not electrically insulated and it therefore makes no sense in these loops to make the short loop section as short as possible in order to hereby make the RF current required for the RF surgical resection and in particular during the incision phase as small as possible.

In order to make the short non-electrically insulated loop section in the resection loop as short as possible with regard to the lowest possible RF current required for the RF surgical resection, without making the shear force required for opening this resection loop too great, a sufficiently long proximal section of the stop wire 15 with the electrical insulation 14 is withdrawn from the distal end of the catheter until the entrainer or movable stop element 33 stops against the stop 37 fixed in the catheter.

In this way, it is also prevented that hot electrical arcs between the short electrically non-insulated loop section 2 and tissue, which are required to cut tissue, thermally damage the distal end of the catheter 12 and in particular that the wire of the short loop section heated by the hot electrical arcs melts into the distal end of the catheter, in particular as long as the short non-electrically insulated loop section is pressed onto the wall of the catheter when the resection loop is opened.

The entrainer device comprises an entrainer 32 fixed on the manipulation wire 12 and the stop element 33 required for the stop device already described above which is connected to the stop wire 15. This entrainer device draws the short loop section into the catheter as soon as the entrainer 32 impacts against the stop element 33 when withdrawing the manipulation wire 11 and therefore also the long loop section 3 into the catheter.

With the asymmetric resection loop, theoretically arbitrarily large polyps of flat lesions of the mucosa of the gastrointestinal tract following submucosal underinjeciton can be resected en-bloc, i.e. in one piece, with low RF current and negligible incision delay near the muscularis propria, where this loop can be pressed against the organ wall during the RF surgical resection. Another advantage of this resection loop is that when used as intended, this only cuts parallel to the organ wall or to the muscularis propria and not in the direction of the organ wall, thus avoiding perforations of the organ wall.

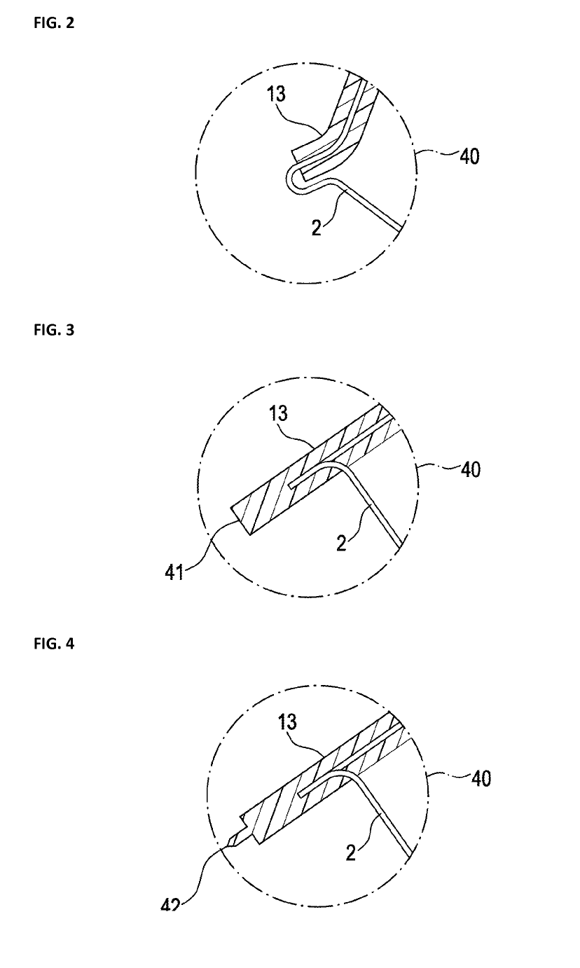

In order to prevent this loop cutting into the organ wall, for example, when not used as intended or accidentally, the resection loop, as shown schematically in FIG. 3 can be fitted at its distal end 40 with an electrically non-conductive spur 41 which is so long that it prevents any contact of the short loop section, for example, with the organ wall as is possible with the distal end of the loop shown schematically in FIG. 2.

In order to be able to press the resection loop in a manner as controlled as possible and sufficiently firmly against the organ wall around polyps or flat lesions when this is applied around polyps or flat lesions of the mucosa to be resected, this resection loop, as shown schematically in FIG. 4, is fitted with an electrically non-conductive mandrel 42 at its distal end 40, which can be inserted into the mucosa quasi as a pivot point for the resection loop in the vicinity of a polyp or a flat lesion of the mucosa in order thereafter to tilt the opened loop about this pivot point above the polyp or the flat lesion. A torsionally rigid manipulation wire which has already been mentioned above can be used for this . . . .

It will be appreciated to those skilled in the art having the benefit of this disclosure that this invention is believed to provide RF surgical resection instruments having a resection loop for removal of pathological tissue. Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

### LIST OF REFERENCE NUMERALS

2 Short electrically non-insulated loop section
3 Long electrically insulated loop section
10 Loop or resection loop
11 Manipulation wire
12 Catheter
13 Electrical insulation of the electrically long loop section 3
14 Electrical insulation on the stop wire 15
15 Stop wire
32 Entrainer
33 Stop element which is movable in the catheter
35 Hole
37 Fixed stop in the catheter
40 Distal end of the resection loop 10
41 Spur
42 Mandrel
50, 51 Opening width of the resection loop 10

1. A radio frequency (RF) surgical resection instrument comprising
an asymmetric opening and closing resection loop configured to ensnare and RF surgically separate tissue, the loop comprising:
an electrically non-insulated shorter loop section having a proximal end, a distal end, and a first length, and
an electrically insulated longer loop section having a proximal end, a distal end, and a second length,
wherein the distal ends of the shorter and longer loop sections are at least one of mechanically and electrically connected to each other, and the first length is a minority of the second length,
a catheter comprising an electrically non-conductive material, the catheter having a proximal end, a distal end, and a longitudinal opening extending between the first and second ends,
a manipulation wire disposed in the longitudinal opening of the catheter, the manipulation wire having a proximal end and a distal end that is:
at least one of mechanically and electrically connected to the proximal end of the longer loop section, or
formed as an extension of the proximal end of the longer loop section,
wherein the manipulation wire is configured:
to be advanced distally inside the catheter in the longitudinal direction to advance the longer loop section such that the longer loop section pulls the distal end of the shorter loop section distally away from the distal end of the catheter, and
to be withdrawn proximally to retract the longer loop section,
a stop device comprising:
a stop wire having a proximal end and a distal end that is connected to the proximal end of the shorter loop section,
a first entrainer coupled to the stop wire at the proximal end of the stop wire, the first entrainer being movable relative to the catheter,
a stop which is fixed relative to the catheter, inside the catheter, such that the first entrainer will contact the stop when the shorter loop section is pulled distally away from the distal end of the catheter to limit a section of the stop wire that can extend from the distal end of the catheter to a predefined length, and
electrical insulation disposed on at least the section of the stop wire which can extend from the distal end of the catheter such that the insulation extends from the distal end of the catheter when the first entrainer contacts the stop,
a second entrainer which is connected to a distal portion of the manipulation wire, the first and second entrainers configured such that upon withdrawal of the manipulation wire, the second entrainer will contact the first entrainer to also withdraw the stop wire.

2. The monopolar RF surgical resection loop according to claim 1, characterized in that the distal end of the loop is formed as a spur.

3. The monopolar RF surgical resection loop according to claim 1, characterized in that the distal end of the loop is formed as a mandrel.

4. RF surgical resection instrument comprising a monopolar RF surgical resection loop according to claim 1 and a handle at a proximal end of the catheter.
